(19) United States
(12) Patent Application Publication
(10) Pub. No.: US 2023/0309993 A1
(43) Pub. Date: Oct. 5, 2023

(54) ELECTROSURGICAL INSTRUMENT WITH PERMANENT TENSIONER

(71) Applicant: Erbe Elektromedizin GmbH, Tuebingen (DE)

(72) Inventor: Dietmar KARWEI, Ofterdingen (DE)

(21) Appl. No.: 18/191,114

(22) Filed: Mar. 28, 2023

(30) Foreign Application Priority Data
Mar. 31, 2022 (EP) 22166100.2

(51) Int. Cl. A61B 17/068

(52) U.S. Cl. CPC A61B 17/068; A61B 2017/07214

(57) ABSTRACT

The electrosurgical instrument includes an electrode and an insulator arrangement. The latter includes an instrument hose and an insulator arrangement arranged at its distal end. In the instrument hose a longitudinal thrust element is arranged for defining the position of the electrode relative to the distal end of the insulator arrangement. The instrument comprises a stop and a counter stop, whereby the counter stop is assigned to the electrode and/or the thrust element. An elastically deformable device is configured to pretension the counter stop in a distal direction against the stop in order to prevent a movement of the electrode in a proximal direction relative to the distal end of the insulator arrangement and/or the instrument hose during bending or curving of the instrument hose.

### CROSS REFERENCE TO RELATED APPLICATION

This application claims the benefit of European Patent Application No. 22166100.2, filed Mar. 31, 2022, which is incorporated herein by reference in its entirety.

### TECHNICAL FIELD

The invention refers to an instrument having a hose in which a longitudinal thrust element extends, which is coupled with an electrode at the distal end of the thrust element.

### BACKGROUND

A treatment apparatus for an endoscope is known from DE 10 2006 006 052 A1, which comprises a sheath that can be inserted into an insertion channel of an endoscope and comprises an electrically conductive operating wire that can be pushed forward and pulled backward inside the flexible sheath. The electrode shall be releasable from the high frequency treatment apparatus, independent from the operating wire. In the treatment apparatus a spring can be arranged that applies an expansion force on the electrode acting in direction of the proximal end of the electrode. By means of the operating wire the electrode can be moved through a hole in the distal end of the treatment apparatus, so that the electrode projects a bit over the distal end of the sheath out of the hole.

In such instruments the length of the part that extends beyond the distal end of the flexible sheath can depend on the curvature or bend of the sheath. In some instruments this can be traced back to a gap that is left empty between the fixed flexible sheath and the operating wire. The gap can emerge from diameter tolerances of the components, for example, and can also be provided in order to provide a necessary play between the components for the extension and retraction movement. Apart from that plastic is usually used for the flexible sheath of such an instrument that may deform under application of force. As a result, if the electrode tip projects a certain length beyond the distal end of the sheath in the neutral stretched position of the instrument, it can no longer be guaranteed that the electrode tip still has this distance from the distal end of the sheath with the instrument being bent or curved.

This circumstance is taken into account in WO 2009/030 324 A1, in that at the end of the protection hose of the instrument and elastomer ring is arranged, which surrounds the needle electrode. The elastomer ring is configured so that a defined holding force is induced that fixes the needle electrode in the position adjusted beforehand. However, during forward and back movement of the needle electrode this friction force for fixing the needle electrode has to be always overcome.

### SUMMARY

It is one object of the invention to provide an instrument having an improved concept, particularly for maintaining a distance of an electrode tip from the distal end of an insulator.

This object is solved by means of an electrosurgical instrument as described herein.

The electrosurgical instrument according to the invention comprises an electrode, e.g. made of tungsten, and an insulator arrangement, which can comprise an instrument hose and an end piece. The end piece can be an insulating body consisting of ceramic. In the instrument hose a longitudinal (e.g. an inner hose or wire) can be arranged provided for changing the position of the electrode, particularly the electrode tip relative to the distal end of the insulating arrangement, e.g. for forwarding the electrode beyond the end of the insulating arrangement.

The instrument comprises a stop that can be formed, for example, in or on the insulator body. In addition, the instrument comprises a counter stop of the electrode or the thrust element. An elastically deformable device, e.g. a spring elastic element (spring element) is arranged and configured to pretension the counter stop in distal direction against the stop in order to keep or fix the position of the electrode, particularly the position of the electrode tip relative to the distal end of the insulating arrangement and/or relative to the distal end of the hose, during bending or curving of the hose. An undesired movement of the electrode in proximal direction, particularly a movement of the electrode tip in proximal direction relative to the distal end of the insulating arrangement and/or the instrument hose is thus effectively avoided, also in case of sharp bending or curving of the instrument hose.

The thrust element extends from the distal end of the instrument up to its proximal end, so that the movement of the electrode is possible due to the influence on the proximal end of the instrument. The thrust element can be an electrical conductor, electrically connected with the electrode for application of the electrode with electrical high frequency power. For example, the electrode can be used for cutting of biological tissue. The thrust element can be a shear-resistant inner hose arranged inside the lumen of the outer instrument hose. In or on this shear-resistant inner hose in turn an electrical conductor, e.g. a wire, can be arranged, which extends through the inner hose and serves for current supply of the electrode.

The outer instrument hose is made of electrically insulating material, e.g. plastic. Preferably also the inner hose is made of plastic.

The outer diameter of the thrust element is preferably smaller than the inner diameter of the instrument hose in order to provide a transverse play for the largely force-free forward movement and preferably retraction of the thrust element in the instrument hose.

The stop is preferably immovably fixed relative to the distal end of the insulating arrangement and/or the distal end of the instrument hose. The stop defines a position of the counter stop pretensioned against the stop relative to the end of the insulating arrangement and/or relative to the instrument hose.

The counter stop is coupled with the electrode and/or the thrust element, particularly immovably fixed or formed on the electrode and/or the thrust element. The counter stop and the electrode and/or the thrust element are immovably relative to one another. The counter stop can be formed, particularly attached by forming, on the electrode, a base element or the thrust element. The counter stop can also be realized by a separate element that is attached on the electrode, a base element or the thrust element.

The elastically deformable device can be the thrust element or a section thereof, which is configured, particularly formed and provided to be elastically deformed during forward movement of the electrode in order to pretension the counter stop in distal direction against the stop. Alternatively or additionally, the elastically deformable device can be or comprise a spring element, e.g. a pressure spring, which is separate from the thrust element. The elastically deformable device provides an elastic length reserve (length storage). Due to this elastic length reserve, the distal end of the electrode can always be kept in a defined position relative to the distal end of the insulating body, particularly in distal extension position in all curved or bent conditions of the instrument hose occurring during surgical intervention with the instrument. The length of the portion of the electrode that protrudes from the distal end of the insulating body and/or the hose is thus independent from the curvature or bend of the instrument hose. The thrust element can be realized by a shear-resistant wire, particularly a metal wire. It can also serve for current supply of the electrode. The thrust element can also be realized by a shear-resistant additional hose, which is arranged inside the lumen of the instrument hose. This additional hose can be connected with the electrode an electrode base serving as counter stop element. The electrode and the electrode base can comprise a central channel to which gas or liquid can be supplied via the additional hose.

The insulating body forms the distal end of the instrument. Preferably, the stop is formed on or in the insulating body. The insulating body preferably comprises a tube-like extension on which the outer instrument hose is held. The stop can be arranged on the distal end of the tube-like extension or can be formed by a ring shoulder that is realized inside the channel extending through the tube-like extension.

The elastically deformable device, e.g. the spring element, can be arranged proximally outside of the hose, e.g. adjacent thereto. A handle can adjoin the proximal end of the instrument hose. The elastically deformable device, e.g. the spring element, can be arranged in the handle.

Preferably, the stop is arranged at the distal end of the instrument, e.g. in or on the insulating body arranged at the distal end in the proximity of the electrode. The elastically deformable device is preferably effective between the proximal operating means and the stop. Thereby each length difference between the instrument hose and the thrust element, which expands the electrode, occurring due to a movement or curvature or bend of the instrument, can be compensated.

The insulating body is preferably held by the instrument hose. It can be attached by means of friction fit between the stop element and the instrument hose inside the latter. Preferably, the connection between the insulating body and the instrument hose is not substance bonded. Alternatively or additionally, the connection between the insulating body and the instrument hose is preferably not form-fit, but preferably only friction-fit.

Preferably, the insulating body projects beyond the distal end of the instrument hose and preferably forms the distal end of the insulating arrangement. Preferably, the insulating body consists of ceramic or another insulating material.

The counter stop can be formed by a counter stop element, particularly by its distal end. The counter stop element can be an element separate from the electrode and/or the thrust element. The counter stop element can serve for electrical connection between the electrode and the thrust element and can be configured as electrode base, for example. The counter stop element can consist of stainless steel, for example.

The counter stop element can comprise an end in which the electrode is inserted and another end in which the distal end of the thrust element is inserted. For example, the counter stop can be realized by a face or a shoulder surface of the counter stop element. For example, the counter stop element can consist of stainless steel.

The counter stop element can establish a mechanical shear- and tension-resistant connection between the thrust element and the electrode in order to be able to move the electrode in controlled manner in distal and proximal direction by means of the thrust element. The sleeve can stiffen the arrangement formed by the distal end of the hose and the proximal end of the electrode.

The thrust element forms or contains the electrode supply line for supply of the electrode with electrical power and is preferably made of flexible metal, e.g. nitinol, if the thrust element of flexible metal and an electrode of tungsten are used, a high flexibility of the thrust element on one hand and a high durability of the electrode on the other hand can be achieved. The thrust element of flexible metal and the electrode of tungsten can be connected by means of a sleeve, for example, which can form the counter stop element. Alternatively, the electrode can be a distal end section of the thrust element.

If the thrust element is configured as inner hose, it can be connected with the proximal end of the base element that supports the electrode on the distal side. The base element and the electrode can comprise a passage channel through which gas or liquid can be ejected. The passage channel can be arranged axially centrally.

The instrument according to the invention can comprise a connection element for connection of the instrument hose with an additional hose, independent from the configuration of the thrust element, wherein the additional hose can serve as fluid conduction hose and is guided parallel to the instrument hose. The connection element can be configured seamlessly in one-part form or in multi-part form.

In order to connect the instrument hose with the additional hose at the distal end of the instrument, the connection element can comprise at least two parallel extensions. The instrument hose and/or the additional hose can be plugged on one extension of the connection element respectively. The extension for the instrument hose can comprise a guide channel for the electrode. The extension for the additional hose can comprise a fluid channel that is fluidically connected with the additional hose. The fluid channel can extend up to the distal end of the connection element and can comprise a nozzle section at its distal end.

The connection element is preferably rigid. Preferably, the connection element is less elastic in distal direction than the instrument hose and less elastic than the additional hose. The connection element can form or comprise the stop element.

In a multi-part configuration of the connection element (which can also be denoted as connection device) an extension of a first part and an additional extension of a second part can be formed that is a part separate from the first part. The first and the second part can consist of ceramic respectively. A third part of the connection element, which is separate from the first part and/or the second part, serves for connection of the first part and the second part with one another and establishes the connection of the instrument hose with the additional hose. The third part can be a sheet part, particularly sheet metal part.

The instrument can be guided through a working channel of an endoscope. In embodiments having an additional hose, the instrument hose and the additional hose can be guided through the same working channel of an endoscope or through one individual working channel respectively.

The instrument hose and the additional hose can be arranged in a sheath, particularly in a common lumen of a sheath, which can extend up to the distal end of the hoses. The sheath can end proximally on or in a handle of the instrument. The sheath can be a shrinking hose, which is continuous over the entire length of the hoses or the entire length between the handle and the distal end of the hoses, wherein the shrinking hose surrounds the hoses and is shrunk on the hoses.

An operating part can be connected to the thrust element that is elastically connected with the thrust element in thrust direction. The operating part can be elastically connected with the thrust element, also in proximal direction in order to be able to retract the electrode with different magnitudes into the insulator arrangement. The elastically deformable device can serve for elastic coupling, also in proximal direction. Alternatively, the instrument according to the invention can have an additional elastically deformable device. The latter is preferably effective between an operating part and the thrust element. The instrument can be configured so that in case of a movement of the operating part for retraction of the thrust element or the electrode, an additional spring element as additional elastically deformable device is moved in proximal direction against a stop. The stop can be coupled with the thrust element in order to transfer the movement of the operating part on the thrust element for retraction of the electrode. The additional spring element thereby provides for a length compensation in order to be able to use the device for retraction of the electrode, e.g. needle-shaped and L-shaped electrodes, that can be retracted into the insulator arrangement with different magnitudes.

### BRIEF DESCRIPTION OF THE DRAWINGS

Additional advantages, embodiments and features of the instrument according to the invention are derived from the following description, the dependent claims as well as the figures.

They show by way of example:

FIG. 1—a perspective view of an example of an instrument according to the invention,

FIG. 2—a distal end section of the example of the instrument according to the invention according to FIG. 1,

FIG. 3—the distal end section according to FIG. 2 in a longitudinally cut illustration through the plane that is defined by the center axis of the fluid opening and the center axis of the electrode opening,

FIG. 4—a longitudinal cut through the handle at the proximal end of the example of the instrument according to FIG. 1,

FIG. 5a—a longitudinal cut through the working channel of an endoscope shank through which an instrument hose and an additional hose of an embodiment of the instrument according to the invention extends,

FIG. 5b—a longitudinal cut through two working channels of an endoscope shank, whereby the instrument hose of the embodiment of the instrument according to the invention extends into one working channel and the additional hose of the embodiment of the instrument according to the invention extends into the other working channel.

FIG. 6a—a perspective illustration of a section of another embodiment of the instrument according to the invention with the distal end thereof,

FIG. 6b—a longitudinal cut illustration of a section of the instrument according to FIG. 6a with the distal end thereof,

FIG. 6c—an example of a third part of the connection device for connection of the hoses of the embodiment according to FIGS. 6a and 6b,

FIG. 7—a group having an alternative thrust force transmission element, which can be used in the embodiment according to FIG. 4,

FIG. 8—an instrument in coaxial configuration in a longitudinally cut illustration in part,

FIG. 9—the distal end of the instrument according to FIG. 8,

FIG. 10—a handle for operating the instrument according to FIGS. 8 and 9.

### DETAILED DESCRIPTION OF THE DRAWINGS

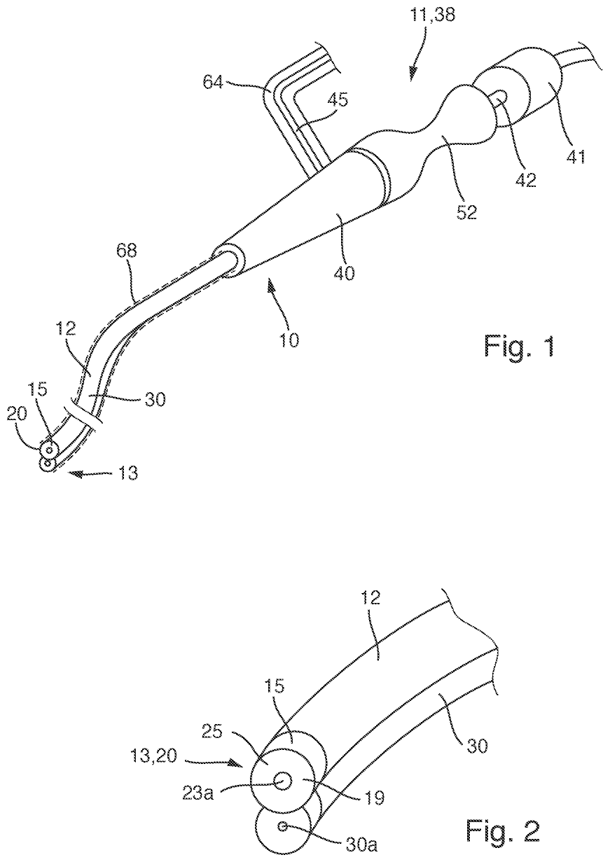

Embodiments of the inventive instrument 10, as exemplarily illustrated in FIGS. 1 to 4, described in the following comprise preferably a handle part 11 (handle) and a flexible instrument hose 12 that extends from the handle part 11 to the distal end 13 of instrument 10.

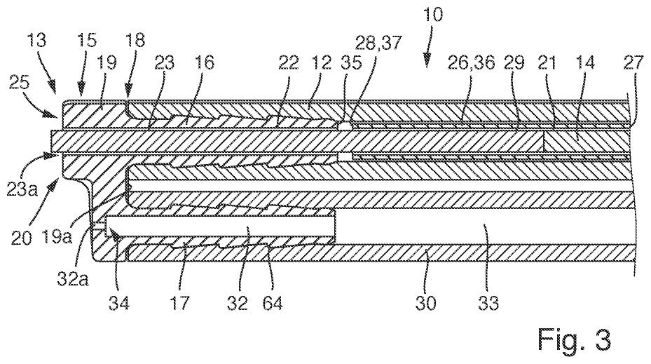

As shown in FIG. 3, a longitudinally movable thrust element 14 extends inside instrument hose 12 consisting of electrically insulating material. The thrust element 14 is preferably electrically conductive or comprises an electrical conductor in order to supply electrode 22 with electrical power. The thrust element 14 can be a metal wire, e.g. made of nitinol.

The outer diameter of thrust element 14 is smaller than the inner diameter of instrument hose 12 and is longitudinally movably guided with transverse play in the instrument hose 12.

An insulating body 15 serving as end piece comprises two tube-like extensions 16, 17 orientated parallel to each other. A distal end 18 of instrument hose 12 is seated on the extension 16 of connection element 15. The connection element 15 and the instrument hose 12 together form an insulating arrangement, the head part 19 of which is the distal end thereof. The connection element 15 and thus the head part 19 and the extensions 16, 17 are preferably seamlessly configured in one single piece. The connection element 15 can be made of ceramic or thermally stable plastic, for example. The instrument hose 12 extends up to the backside 19a facing in proximal direction of head part 19.

Instrument 10 according to the invention can be a monopolar instrument and can comprise accordingly only one electrode 22. The thrust element 14 comprises a distal end 21 that is coupled with the, e.g. needle-shaped, electrode 22. The electrode 22 is longitudinally movably guided inside a channel 23 extending through the distal end 18 of instrument hose 12 as well as the extension 16 and the head part 19. In this manner electrode 22 can be moved out of an exit 23a of channel 23 at the face 25 facing in distal direction. The needle electrode 22, for example consisting of tungsten, can be mechanically and preferably also electrically coupled with thrust element 14 by means of a sleeve 26, particularly a stainless steel sleeve, in order to establish an electrical connection between the thrust element 14 and the electrode 22.

The distal end 21 of thrust element 14 is arranged in a proximal end 27 of sleeve 26. The proximal end 29 of the needle electrode 22 is arranged in a distal end 28 of sleeve 26. The distal end 21 of the thrust element 14 and the proximal end 29 of electrode 22 can be welded to the sleeve 26 in order to at least mechanically, preferably also electrically, effectively couple the thrust element 14 with the electrode 22.

The stainless steel sleeve 26 stiffens the arrangement of the distal end 21 of thrust element 14 and of proximal end 29 of electrode 22. For this reason the stainless steel sleeve 26 ends proximally next to the distal end 21 of thrust element 14 in order to keep the flexibility of instrument 10 largely unaffected.

The second extension 17 is formed adjacent to the first extension 16 on the side of connection element 15. Both extensions 16, 17 extend from the head part 19 of connection element 15 in proximal direction. The exemplary instrument 10 is an instrument 10 having two lumen. The first lumen extends through the instrument hose 12. On the second extension 17 an additional hose 30 is seated having a second lumen. This can channel a fluid, particularly a liquid, for example NaCl-solution, to the distal end 13 of instrument 10.

Instrument hose 12 seated on the first extension 16 can be held in a friction-fit manner or by means of form-fit structures. Likewise the additional hose 30 seated on the second extension 17 can be held in a friction-fit manner or by means of form-fit structure 64 based on the outer surface of second extension 17 and the interior side of the distal end section 31 of the additional hose 30. The connection between the additional hose 30 and the second extension 17 is preferably fluid-tight.

The second extension 17 can also surround a channel 32 that is fluidically connected with a channel 33 limited by the additional hose 30. The channel 33 can comprise a nozzle section at the distal end in which the flow cross-section is narrowed. The channel 32 opens out in an exit 32a, which is arranged on the distal face 25 of connection element 15, just like the exit 23a for the electrode 22.

The first extension 16, onto which instrument hose 12 is plugged, forms a stop with its proximal face 35 in order to define the position of electrode 22 and/or the distal end 21 of thrust element 14 relative to the distal end of the insulator arrangement and/or the distal end of instrument hose 12. The sleeve 26 forms a counter stop element 36, whereby the distal face 37 of sleeve 26 forms a counter stop in order to define the position of electrode 22 and/or the distal end 21 of thrust element 14 relative to the distal end 20 of the insulator arrangement and/or the distal end 18 of the hose. In other embodiments the counter stop can be formed by a thickening of electrode 22 adjacent to its proximal end, for example.

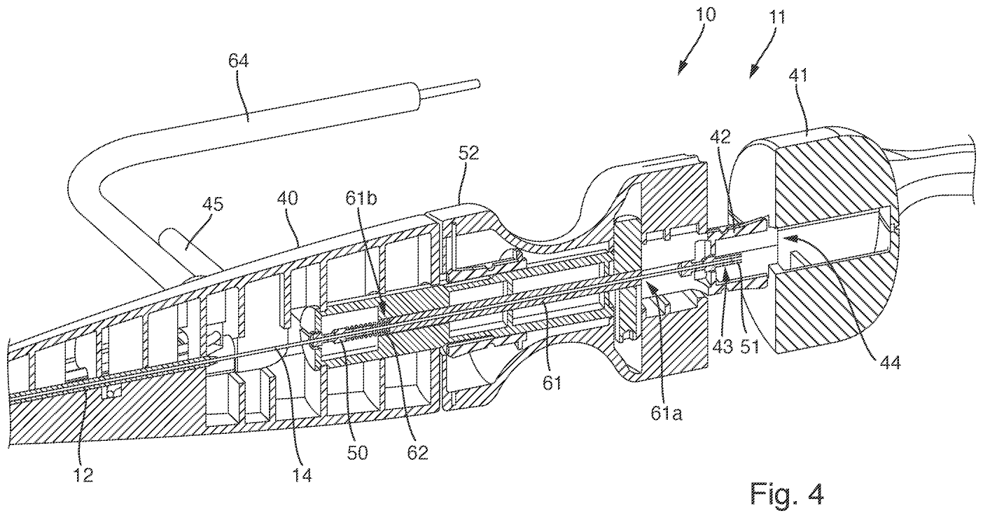

Handle part 11 on the proximal end 38 of instrument 10, which is apparent from FIG. 4, comprises a distal section 40, a proximal section 41 and a shank section 42 that rigidly connects the distal section 40 and the proximal section 41 of handle part 11 with one another. The proximal end section 43 of thrust element 14 extends through the distal section 40 and into shank section 42 in front of its proximal end 44. The instrument hose 12 through which thrust element 14 extends, extends into the distal section 40 of handle part 11 and is fixed there. The thrust element 14 can be electrically connected with an electrical supply line 45 by means of a sliding contact.

A first coupling element 50 is fixed on the thrust element 14. In the illustrated embodiment the first coupling element 50 surrounds thrust element 14. At the proximal end 44 of shank section 42 thrust element 14 is coupled with a second coupling element 51. On the shank section 42 an operating part 52 is arranged in a longitudinally movable manner, which can be operated with the fingers. The operating part 52 can be moved forward from a first position (not illustrated) in which the operating part 52 is arranged on the proximal section 41 into a second position (illustrated in FIG. 4) in which the operating part 52 is arranged on the distal section 40, in order to move electrode 22 forward from a retracted position into a distinct forward position in which the electrode tip 60 has a defined distance to the face 25 of connection element 15 and thus to the distal end 20 of insulator arrangement.

As further illustrated in FIG. 4, the operating part 52 is coupled with a thrust force transmission element 61 for this purpose, which is for example, tube-shaped, through which the thrust element 14 extends in the embodiment, the thrust force transmission element 61 extending movably along shank section 42 inside shank section 42 in the embodiment. Between the thrust force transmission element 61 and the first coupling element 50 a pressure spring 62 is arranged through which the thrust element 14 extends.

The operating part 52 can be blocked in a form-fit manner in the first position and/or in the second position, particularly latched.

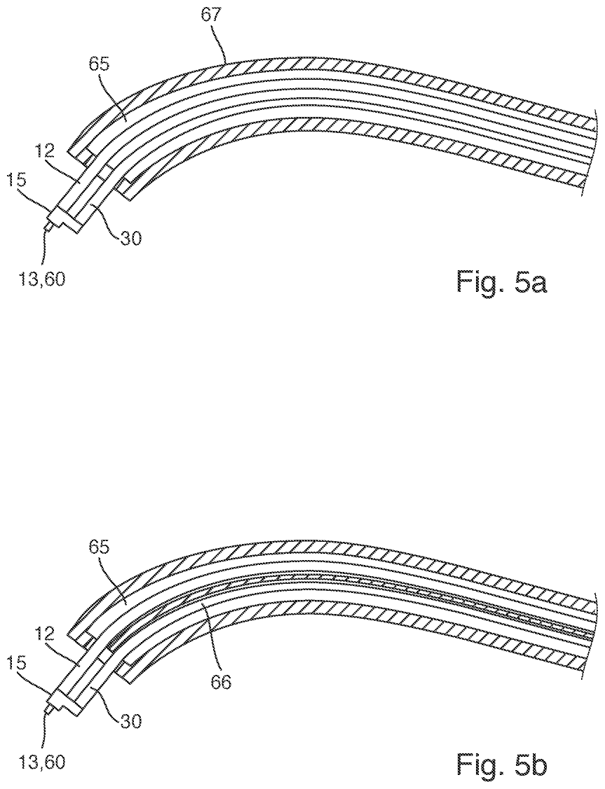

The instrument hose 12 and the additional hose 30 can be guided together through one working channel 65 (see FIG. 5a) or separately through two working channels 65, 66 of an endoscope 67 (see FIG. 5b) respectively.

By means of the instrument 10 according to the invention it can be operated as follows:

Different to the illustration in FIG. 4, an arrangement of the operating part 52 on the handle part 11 of instrument 10 in the first position is assumed. The electrode 22 is accordingly in a retracted position. The distal end of electrode 22 can in this position also project from the distal end of the connection element 15. However, preferably electrode tip 60 (apparent from FIG. 5a) is retracted relative to the exit 23a of the insulator arrangement (exit 23a of connection element 15), through which the electrode 22 can be moved, or ends flush with the exit of the connection element 15.

The instrument 10 can extend through an endoscope (not illustrated), wherein the connection element 15 is arranged outside the endoscope. The user now desires to move the electrode tip 60 forward and indeed in a position having a defined distance of the electrode tip 60 from the exit 23a of the insulator arrangement. As necessary, the user has to unblock the latching of the operating part 52 in the first position and has to move the operating part 52 forward in distal direction relative to the distal section 40, the shank section 42 and the proximal section 41, whereby this forward movement is transferred onto the thrust force transmission element 61. Due to forward movement of operating part 52, finally a force is applied via the thrust force transmission element 61 onto pressure spring 62 in longitudinal direction of pressure spring 62 that transfers the force on the first coupling element 50 and via the latter onto thrust element 14, so that thrust element 14 is moved forward in distal direction and thus also moves the electrode 22 forward in distal direction. The forward movement is preferably possible in a largely friction-free manner. A means used in the prior art for avoiding an undesired movement of the electrode in proximal direction due to friction during curving or bending the hose can be avoided.

If a counter stop 37 of counter stop element 36 abuts against the stop 35 on connection element 15, the operating part 52 has not yet reached the second position. The user moves the second operating part 52 now further forward in distal direction and thereby compresses pressure spring 62 in the direction of the force flow from the operating part 52 toward the counter stop element 36 in order to pretension the counter stop element 36 against the stop element 15. Finally the user brings the operating part 52 in the second position in which the operating part 52 can be latched. The electrode 22 has reached its defined position, if counter stop 37 has established contact with stop 35. The compressed spring 62 forms a force reserve or provides for a length reserve, the force reserve serving to keep the counter stop 37 in abutment with the stop 35, also if the endoscope and/or the instrument hose 12 is bent or curved more or less sharply. The arrangement with the tensioned spring element 62 forms a permanent tensioner for the counter stop 37 against the stop 35. Preferably in all of the surgical applications for which the instrument 10 can be used the counter stop element 36 is kept in abutment with the stop element 15 and thus the position of the electrode tip 60 relative to the distal end 20 of the insulator arrangement is maintained. By means of the pretensioned spring 62, a length compensation is provided in the instrument 10 and the electrode is kept in the projecting position. The elastical pretensioning of counter stop 37 in distal direction is preferably not provided or not exclusively provided by particularly arbitrary, lateral dodging or meandering of thrust element 14 due to the thrust force introduced by means of the operating part 52. Rather the pressure spring 62 forms a separate elastically deformable device that is configured and determined to form a length reserve in the elastically deformed condition. Alternatively or additionally to the spring element 62, particularly a pressure spring, the device can also be formed by a section of the thrust element 14 (not illustrated) that is configured—for example shaped and determined to be elastically longitudinally compressed in forward movement direction during forward movement of the electrode 22 in distal direction. For example, the section can be helically wound. The section is preferably limited in its length on a distinct sub-section of the thrust element. Independent from whether the elastically deformable device is formed by a spring element 62 and/or an elastically deformable section of the thrust element 14, the device is preferably arranged in the handle part 11 of instrument 10.

The user can apply an RF power to the electrode via the supply line 45, the sliding contact and the thrust element 14 in order to carry out a treatment on a patient. For example, the user can cut tissue of the patient by means of the electrode. Alternatively or additionally, the user can channel fluid, particularly NaCl-solution, via a fluid supply line 63 connected with the additional hose 30 into the additional hose 30 and into the channel 32 to the distal end of the connection element 15 and out of the latter. The pressure spring 62 can be relaxed in the retracted condition of electrode 22 (operating part 52 is in first position) and can be highly compressed in the extended condition or the pressure spring 62 can also be elastically deformed in distal direction (pretensioned) already in the first position of the operating part 52.

In order to move the electrode 22 backward, the user can unblock the latching of the operating part 52, whereupon spring 62 relaxes and retracts the electrode 22 by means of the thrust element 14 that is connected with the electrode 22 via sleeve 26. The user can move the operating part 52 back into the second position in which the electrode 22 is retracted as far as possible and can latch the operating part 52 in the second position.

As indicated in FIG. 1 with dashed lines, instrument hose 12 and the additional hose can be arranged in a hollow space of a sheath 68. The sheath 68 can extend from the handle part 11 up to the connection element 15. The sheath 68 can be a single part or monolithic shrinking hose that extends from the handle part 11 up to the connection element 15 and is shrunk on the instrument hose 12 and the additional hose 30. Alternatively, the sheath 68 can be composed from a number of shrinking hose sections that are distributed over the entire length of instrument hose 12 and additional hose 30 and are shrunk thereon. This has the advantage compared with the shrinking hose having a continuous section over the entire length of the hoses 12, 30 that the hoses 12, 30 are more flexible having a sheath configured in this manner.

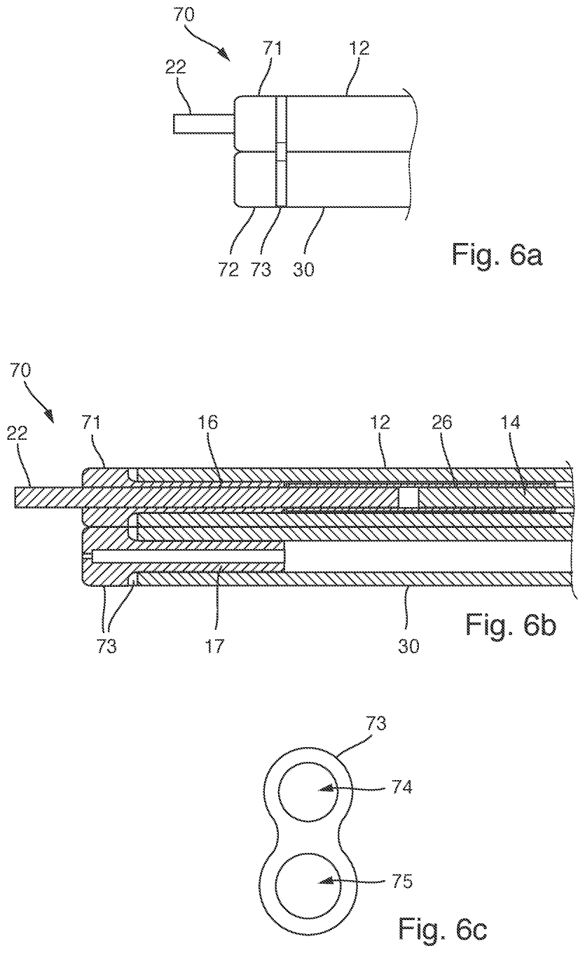

FIGS. 6a to 6b illustrate in part exemplary use of an instrument 10 according to another embodiment. For a connection of the instrument hose 12 and the additional hose 30 a connection device 70 is provided. It comprises a first part 71 having an extension 16 and a second part 72 having an additional extension 17, whereby the two parts 71, 72 are separate parts, e.g. made of ceramic. A third part 73 of the connection device 70, which is separate from the first part 71 and the second part 72, serves for connection of the two parts 71, 72 with one another and thus for connection of the instrument hose 12 and the additional hose 30. The third part 73 can be a flat metal part, for example. The third part 73 comprises at least one opening, preferably, as illustrated in FIG. 6c, one opening 74, 75 for the first part 71 and the second part 72 respectively. The third part 73 can have the shape of an eight. In order to establish a connection between the instrument hose 12 and the additional hose 30, the extension 16 and the first part 71 and the extension 17 and the second part 72 are plugged into the openings 74, 75 of the third part 73. Then the instrument hose 12 and the additional hose 30 are moved onto the extensions 16, 17 of first part 71 or second part 72 respectively. The hoses 12, 30 can be moved against the third part 73. The connection between instrument hose 12 and additional hose 30 is thus established. The outer contour of the third part 73 can correspond to the hose contours. The proximal face 35 of extension 16 of first part 71 forms a stop for the face 37 on the distal end 28 of the sleeve 26, how and for which purpose is described in connection with the embodiment according to FIGS. 1 to 4.

In the embodiment illustrated in FIG. 4 it is possible to retract operating part 52 so far until it abuts against the proximal section 41. In the course of this movement the proximal face 61a of thrust force transmission element 61 is urged against the second coupling element 51, so that the needle electrode 22 is accordingly far retracted into the channel 23 of connection element 15. The instrument 10 can comprise an electrode that cannot be completely retracted into the channel 23, because the dimension of the electrode at its distal end is too large for the channel 23. For example, the electrode can be an L-shaped electrode or a needle-shaped electrode having a ring around the needle. If it is now attempted to move the operating part 52 further in proximal direction, thereby potentially a too high force can be transferred from the operating part 52 on the electrode, which can be damaged thereby. An additional spring element 76 arranged between the proximal face 61a of the thrust force transmission element 61 and the second coupling element 51 avoids this. The second coupling element 51 is then accordingly arranged offset toward the proximal end of the thrust element 14. For example, thrust element 14 can extend through an additional pressure spring 76 that is arranged between the thrust force transmission element 61 and the second coupling element 51 (not illustrated in FIG. 4). During a movement of the operating part 52 for retraction of the thrust element 14 or the electrode, the additional pressure spring 76 is moved in proximal direction against the second coupling element 51 coupled with the thrust element 14 in order to transmit the movement of the operating part 52 for retraction of the electrode onto the thrust element 14. If the electrode and/or the thrust element 14 can no longer be retracted further into the channel and/or the hose, the second spring element 76 is elastically deformed during a further movement of the operating part 52. This results in a limitation of the force onto the electrode that is for example L-shaped, or the thrust element 14.

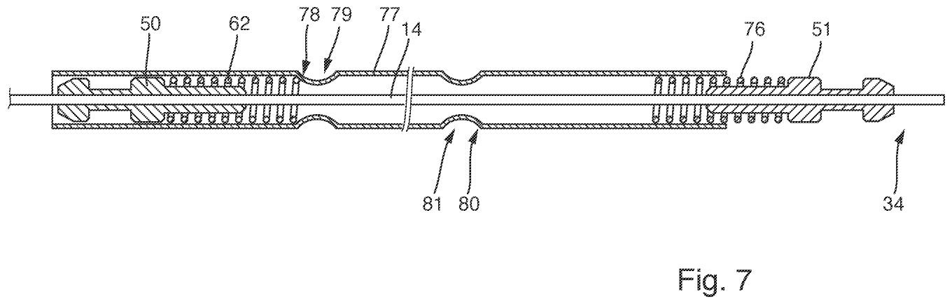

An additional spring element 76 serving for the purpose described above is illustrated in FIG. 7. FIG. 7 also shows an alternative thrust force transmission element 77, which can be used instead of the thrust force transmission element 61 according to FIG. 4 in the instrument 10. The thrust force transmission element 77 according to FIG. 7 is configured to limit a lateral dodging (transverse to the longitudinal extension direction of the thrust element 14) of elements arranged in the thrust force transmission element 77. The thrust force transmission element 77 surrounds the first coupling element 50 and the pressure spring 62 and limits a dodging of pressure spring 62, the first coupling element 50 and/or the thrust element 14. Likewise the thrust force transmission element 77 surrounds the second coupling element 51 and the additional pressure spring 76 and limits dodging of the additional pressure spring 76, the second coupling element 51 and/or the thrust element 14 during retraction of the electrode 22. The thrust force transmission element 77 can be configured as thin-walled tube 77, particularly stainless steel tube. The ends of the tube surround the pressure springs 62, 76 and the coupling elements 50, 51. The thrust force transmission element 77 comprises a stop section 78, which is formed by a groove 79 in the illustrated embodiment. The stop section 78 is urged against the pressure spring 62 during forward movement of the thrust force transmission element 77. In this manner the force is transmitted from the operating part 52 coupled with the thrust force transmission element 77 in distal direction via the first coupling element 50 on the thrust element 14. The first stop section 78 of the thrust force transmission element 77 in so far takes the function of the distal face 61b of the thrust force transmission element 61 of FIG. 4. The thrust force transmission element 77 comprises in addition a second stop section 80, which is formed by a second groove 81 in the illustrated embodiment. The stop section 80 is urged against the additional pressure spring 76 during retraction of the thrust force transmission element 77. The additional pressure spring 76 transmits the force from the operating part 52 on the thrust element 14 in order to retract electrode 22. The second stop section 80 in so far takes the function of the proximal face 61a of the thrust force transmission element 61 of FIG. 4. Alternatively to the embodiments having a spring element 62 for length compensation and distal direction and an additional spring element 76 for length compensation in proximal direction, also respective length compensations can be provided with only one elastically deformable device configured and arranged to be elastically deformed in two directions, particularly one spring element. In addition, the thrust element 14 can be tensile elastically configured and serve as tensile spring element.

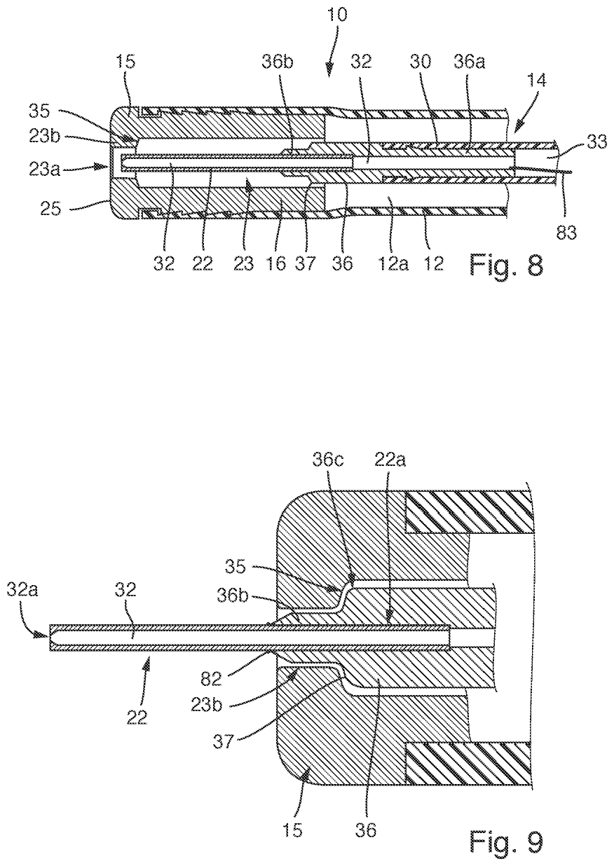

FIG. 8 shows a modified embodiment of instrument 10. The above explanation applies for this embodiment on the basis of similar reference signs with regard to the structure as well as with regard to the function under consideration of the following explanations accordingly.

Different to the instrument according to FIGS. 1 to 7 described above, the instrument 10 illustrated in FIGS. 8 and 9 is a co-axially configured instrument having a hollow electrode 22, which comprises an exit opening 32a at its distal end. The channel extending through the electrode 22 is in fluid connection with the additional hose 30 extending through the lumen 12a up to the handle part 11. The instrument hose 30 is somehow shear-resistant, so that it is indeed bendable, but thereby definitely suitable for transmission of thrust forces. The counter stop element 36 being here configured as electrode base can serve for connection between instrument hose 30 and electrode 22 in that the proximal end 22a of electrode 22 is held, as FIG. 9 illustrates. The counter stop element 36 comprises a shank 36 extending in proximal direction into the additional hose 30, wherein the shank 36 is connected with the additional hose 30 in a tensile and thrust rigid manner and through which a passage extends connecting the channel 33 with the channel 32.

The stop element 35 comprises a ring shoulder inside its channel 23 facing the counter stop element 36 serving as stop 15 for the counter stop element 36. Starting from the ring shoulder, channel 23 narrows to the narrowed section 23b.

The counter stop element 36 comprises an extension 36b fitting through the narrowed section 23b, wherein the outer diameter of the extension 36b is smaller than the outer diameter of the remaining counter stop element 36. The counter stop element 36 comprises accordingly a ring shoulder 36c that gets into abutment with stop 35, if the electrode 22 takes the completely extended position. For connection of the electrode 22 with the counter stop element 36 one or multiple welded joints 82 can be provided, which is or are provided on the part of the section 36b of counter stop element 36 that projects beyond the distal face 25 in the extended position.

The instrument hose 12 and the additional hose 30 can also be made of plastic. For current supply of electrode 22 an electrical conductor 83 can be provided extending through the channel 33 of additional hose 30. The electrical conductor 83 can be electrically connected with the shank 36a of counter stop element 36, e.g. by means of welding. It is also possible to integrate the electrical conductor 83 in the plastic of the additional hose 30 or to guide the electrical conductor 83 inside the lumen 12a of the instrument hose 12. In all of the cases of the co-axial instrument configuration according to FIG. 8 the electrical conductor 83 can either be tensile and thrust rigid itself and thus form part of the thrust element 14. Alternatively, it can be configured in a bendable manner, so that the thrust element 14 is exclusively realized by the additional hose 30.

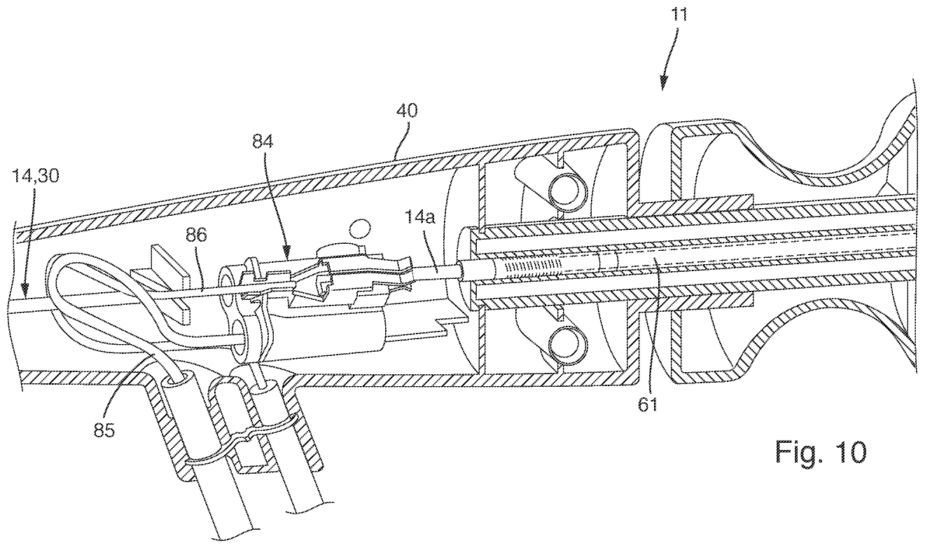

FIG. 10 illustrates a slightly modified handle part 11 for operating the instrument 10 according to FIGS. 8 and 9. In terms of the operation of the instrument, the description above applies accordingly with the following supplement:

The additional hose 30 serving as thrust element 14 leads to a fluid switch 84 that is axially movably arranged in the distal section 40 of handle part 11 configured as housing. Connected to the fluid switch 84 is a fluid supply hose 85, which is fluidically connected with the additional hose 30 via a channel formed in the fluid switch 84. In addition, the fluid switch 84 establishes a mechanically tensile and thrust rigid connection between the additional hose 30 and the proximal thrust element section 14a that extends as tensile and thrust rigid pin or wire into the thrust force transmission element 61. Preferably the fluid switch 84 consists of metal. An electrical flexible supply wire 86 can be connected to the fluid switch 84 on the outer side. The conductor 83 guided through the additional hose 30 can be electrically contacted with the fluid switch 84 inside the latter. Apart therefrom the description provided above in connection with FIG. 4 applies accordingly.

The electrosurgical instrument 10 comprises an electrode 22 and an insulator arrangement. The latter comprises an instrument hose 12 and an insulator arrangement arranged at its distal end. In the instrument hose 12 a longitudinal thrust element 14 is arranged for defining the position of the electrode 22 relative to the distal end of the insulator arrangement. The instrument 10 comprises a stop 35 and a counter stop 37, whereby the counter stop is assigned to the electrode 22 and/or the thrust element 14. An elastically deformable device (spring 62) is configured to pretension the counter stop 37 in distal direction against the stop 35 in order to avoid a movement of the electrode 22 in proximal direction relative to the distal end 20 of the insulator arrangement and/or the instrument hose 12 during bending or curving of the instrument hose 12.

### LIST OF REFERENCE SIGNS

10 instrument
11 handle part
12 instrument hose
12a lumen of instrument hose
13 distal end of instrument
14 thrust element
14a thrust element section
15 connection element/stop element
16 first extension/extension
17 second extension/additional extension
18 distal end of hose
19 head part
19a back side
20 distal end of insulator arrangement
21 distal end of thrust element
22 needle electrode
22a proximal end of electrode 22
23 channel
23a exit
23b narrowed section of channel 23
25 face
26 sleeve
27 proximal end of sleeve
28 distal end of sleeve
29 proximal end of needle electrode
30 additional hose
31 distal end section
32 channel
32a exit, exit port
33 channel
34 nozzle section
35 proximal face/stop
36 counter stop element
36a shank
36b section of counter stop element 36
36c ring shoulder on counter stop element 36
37 distal face/counter stop
38 proximal end of instrument
40 distal section
41 proximal section
42 shank section
43 proximal end section of thrust elements
44 proximal end of shank section
45 electrical supply line
50 first coupling element
51 second coupling element
52 operating part
60 electrode tip
61 thrust force transmission element
61a proximal face
61b distal face
62 pressure spring
63 fluid supply line
64 form-fit structure
65 working channel
66 working channel
67 endoscope
68 sheath
70 connection device
71 first part
72 second part
73 third part
74 opening
75 opening
76 additional pressure spring
77 thrust transmission element/tube
78 first stop section
79 first groove
80 second stop section
81 second groove
82 welded joint
83 conductor
84 fluid switch
85 fluid supply hose
86 supply wire or supply braid

1. An electrosurgical instrument comprising:
an electrode;
an insulator arrangement having a distal end;
an instrument hose in which a longitudinal thrust element is arranged for defining a position of the electrode relative to the distal end of the insulator arrangement;
a stop;
a counter stop of the electrode and/or the longitudinal thrust element; and
an elastically deformable device, which is arranged and configured to pretension the counter stop in a distal direction against the stop in order to avoid movement of the electrode in a proximal direction relative to the distal end of the insulator arrangement and/or the instrument hose when bending or curving the instrument hose.

2. The electrosurgical instrument according to claim 1, wherein the stop is arranged next to a distal end of the instrument hose and/or the insulator arrangement.

3. The electrosurgical instrument according to claim 1, wherein the elastically deformable device is arranged proximally outside the instrument hose and/or wherein the electrosurgical instrument comprises a handle, wherein the elastically deformable device is arranged in the handle.

4. The electrosurgical instrument according to claim 1, wherein the stop is formed by a stop element, which is positioned in a distal end section of the instrument hose and is held in a form-fit and/or friction-fit manner.

5. The electrosurgical instrument according to claim 1, wherein the counter stop is configured on the electrode and/or the longitudinal thrust element and/or on a counter stop element which is connected with the electrode and/or the longitudinal thrust element.

6. The electrosurgical instrument according to claim 1, wherein the counter stop is formed by a counter stop element which is separate from the electrode and the longitudinal thrust element and is operable to connect the longitudinal thrust element and the electrode.

7. The electrosurgical instrument according to claim 5, wherein the counter stop element comprises an extension that extends up to or distally beyond a distal end surface of a stop element comprising the stop.

8. The electrosurgical instrument according to claim 1, wherein the longitudinal thrust element is formed by an additional hose, which is arranged inside the instrument hose.

9. The electrosurgical instrument according to claim 1, wherein the longitudinal thrust element is a thrust-resistant wire.

10. The electrosurgical instrument according to claim 8, wherein an electrical conductor, which is electrically connected with the electrode, is arranged in or on the additional hose.

11. The electrosurgical instrument according to claim 6, wherein the counter stop element is a sleeve in which the electrode is attached on one side and the longitudinal thrust element is attached on another side.

12. The electrosurgical instrument according to claim 4, wherein the stop element comprises a stop section against which the instrument hose and/or an additional hose is allowed to be moved in order to define a position of the distal end section of the instrument hose and/or the additional hose at the stop element.

13. The electrosurgical instrument according to claim 1, further comprising a connection element having a first extension which includes form-fit structures which allow movement of an additional hose on the first extension with a lower force than a force required to detach the additional hose from the first extension.

14. The electrosurgical instrument according to claim 13, wherein the connection element comprises a second extension, wherein the second extension of the connection element is positioned in the instrument hose and is free of form-fit structures on an outer side thereof.

15. The electrosurgical instrument according to claim 1, wherein the instrument hose is configured to be guided through a working channel of an endoscope and/or wherein the instrument hose and an additional hose are configured to be moved through a same working channel of an endoscope or through separate working channels.
